(19) United States
(12) Patent Application Publication
(10) Pub. No.: US 2004/0199073 A1
(43) Pub. Date: Oct. 7, 2004

(54) Method and apparatus for measuring motion of a body in a number of dimensions

(75) Inventor: Xin Ma, Singapore (SG)

Correspondence Address: OSTROLENK FABER GERB & SOFFEN, 1180 AVENUE OF THE AMERICAS, NEW YORK, NY, 100368403

(73) Assignee: Agency for Science, Technology and Research

(21) Appl. No.: 10/406,666

(22) Filed: Apr. 3, 2003

(51) Int. Cl. A61B 5/05

(52) U.S. Cl. 600/424; 600/595

(57) ABSTRACT

A system for detecting motion of a body in a number of dimensions comprises an array of one or more reflective elements attachable to said body such that said reflective elements move with said body, an array of sensors for sensing light reflected from said array of reflected elements on illumination of said reflective elements by a light source, said sensors being adapted to generate output signals corresponding to motion of said body, and a processor for processing said output signals to determine motion of said body in a number of dimensions.

The present invention relates to a method and apparatus for measuring motion of a body in a number of dimensions, preferably, two orthogonal dimensions. In particular, it relates to an optical tracking method and apparatus for so doing, preferably for use with a catheter and guide wire in interventional radiology (IR) procedures.

### BACKGROUND OF THE INVENTION

In conventional techniques, the motion tracking of a plurality of catheters and guide wires used in medical devices and instrumentation for vascular and interventional radiology is performed separately by a number of individual measuring units, one for each catheter and each guide wire. This requires the guide wires to be longer than the catheters thereby increasing the difficulty of manipulation. Furthermore, the tracking signal may be unstable in such conventional systems.

U.S. Pat. No. 4,726,772 describes a medical simulator for enabling demonstration, trial and test of the insertion of torsionally stiff elongated members into small body passages that branch from main passages. Such torqueable members may be guide wires or catheters which are constructed to cause the distal tip to turn or twist in response to a corresponding motion applied by the operator to a proximal portion of the device.

U.S. Pat. No. 4,907,973 is directed to a medical investigative system in which a person interacts with the system to insert information. The information is utilised by the system to establish non-restricted environmental modelling of the realities of the surrogate conditions to be encountered with invasive or semi-invasive procedures. This is accomplished by a video display of simulated internal conditions that appear life-like, as well as by display of monitor data including, for example, blood pressure, respiration, heart beat rate and the like.

The tracking systems of U.S. Pat. No. 4,726,772 and U.S. Pat. No. 4,907,973 are almost the same in that flexible canulations are used to simulate the blood vessels or trachea. Some tactile sensors are fixed along the canulations. In this way, when implements move in canulations, the tactile sensors detect the position of the implements. The weakness of this technology is that the sensors are installed at separate points. As a result, the tracking information is not continuous. Thus, these kinds of tracking systems cannot fulfil the demands of today's exact surgical simulators.

U.S. Pat. No. 6,062,865 is directed to a system for producing highly realistic, real-time simulated operating conditions for interactive training of persons to perform minimally invasive surgical procedures involving implements that are inserted and manipulated through small incisions in the patient. The virtual environment for this training system includes a housing with a small opening. An implement simulating a surgical implement is inserted into the opening and manipulated relative to the housing. A movement guide and sensor assembly monitors the location of the implement relative to the housing and provides data about the implement's location and orientation within the housing. The reported data is interpolated by a computer processor, which utilises a database of information representing a patient's internal landscape to create a computer model of the internal landscape of the patient. With reference to this computer model, the processor controls the occurrence of force feedback opposing the motion of the implement. A two-dimensional image representing the implement as it would appear within the patient is generated by a processor-controlled video imaging system based on the computer model of the patient's internal landscape. This computer image of the implement is then merged with a video image loop of a patients internal landscape as it appears through a heart beat and breathing cycle, and the merged image is displayed on a video display. The combined elements of real-time visual representation and interactive tactile force feedback provide a virtual training simulation with all elements of actual operation conditions, in the absence of a live patient. Optical encoders are used to detect the translation and rotation motion of the catheters and guide wire. In this system, it is difficult to simulate several catheters and guide wire at the same time. Also, as the devices have to be contained in a housing, the whole housing is quite lengthy.

U.S. Pat. No. 6,038,488 is directed to a device for tracking the translational and rotational displacement of an object having two degrees of freedom using a single point of contact with the object. The device is particularly useful in a catheter simulation device for surgery and interventional radiology applications. A spherical contact member is mounted for free rotation about all axes in force-transmitting contact with the surface of the object and a pair of shafts are mounted in tangential engagement with the spherical contact member to reflect the displacement imparted to the object relative to a reference position. This arrangement provides simultaneous tracking of the combined translation and rotational displacement of the object. Measuring the displacement of the object and a haptic applicator are included such that a load may be applied to the object to control precisely the degree of force required to cause displacement of the object. The actual forces applied to displace the object are also measured such that the device is capable of providing a realistic force reflection to simulate the feel of a surgical procedure. A computerised control system and conventional recording device are employed to provide a programmed procedure which provides realistic “feel” to a user of an actual surgical procedure. The device is readily adaptable for interfacing with a virtual reality type programme to provide simultaneously a visual simulation of the surgical procedure.

In U.S. Pat. No. 6,038,488, a mechanism with a rolling ball and two optical encoders is used for motion tracking. The problem with this design is that the unstable contact between the rolling ball and the optical encoder will cause loss of motion signal.

The present invention aims to overcome or ameliorate the abovementioned disadvantages in the prior art systems.

### SUMMARY OF THE INVENTION

According to a first aspect there is provided a method for detecting motion of a body in a number of dimensions comprising the steps of:

(a) attaching an array of one or more reflective elements to said body such that said reflective elements move with said body;

(b) illuminating said array of reflective elements with light from a light source;

(c) sensing said light reflected from said array of reflected elements with an array of sensors for generating output signals corresponding to motion of said body; and

(d) processing said output signals to determine motion of said body in a number of dimensions.

According to a second aspect there is provided a system for detecting motion of a body in a number of dimensions comprising:

(a) an array of one or more reflective elements attachable to said body such that said reflective elements move with said body;

(b) an array of sensors for sensing light reflected from said array of reflected elements on illumination of said reflective elements by a light source, said sensors being adapted to generate output signals corresponding to motion of said body; and

(c) a processor for processing said output signals to determine motion of said body in a number of dimensions.

Further preferred features are set out in the appended claims.

### BRIEF DESCRIPTION OF THE DRAWINGS

The present invention will now be described by way of example and with reference to the accompanying drawings in which:

FIG. 1 is a schematic of four optical sensors for detecting reflective optical signals from a number of reflective elements mounted on a substrate;

FIG. 2 is a graph showing the relationship of the output of one of the sensors of FIG. 1 with the reflective area observed by the sensor;

FIG. 3 is a schematic showing the compensation by two of the sensors in FIG. 1 relative to movements in the reflective area;

FIG. 4 is an illustration of the waveform of the sum of the outputs of two of the sensors of FIG. 1 together with a rectangular waveform obtained therefrom;

FIG. 5 shows the waveforms of the sum of the outputs of pairs of the sensors shown in FIG. 1;

FIG. 6 is a schematic showing the navigation of a catheter and guide wire in operation; and

FIG. 7 is a schematic showing a catheter and guide wire carrying reflective surfaces.

### DETAILED DESCRIPTION OF THE PREFERRED EMBODIMENTS

FIG. 1 shows a substrate 2 whose displacement is to be measured, an array of reflective elements 4, and an array of four photosensors 6a, 6b, 6c and 6d (also denoted as S1, S2, S3 and S4 respectively) for detecting reflective optical signals. The reflective elements 4 have a substantially rectangular peripheral shape and are mounted on the substrate 2, preferably in uniformly spaced and aligned rows and columns. The photosensors 6a-6d for detecting movement of the substrate 2 are mounted on a fixed body independent of the substrate 2 and are spaced therefrom.

The sensors 6a-6d are arranged in two pairs, the first pair being S1 and S2 (6a and 6b), the second pair being S3 and S4 (6c and 6d). Each sensor in each pair is laterally spaced from the other sensor, and the sensors 6a-6d are arranged to form an array having a substantially rectangular peripheral shape, with a sensor arranged in each corner of the rectangle. Preferably, the pairs of sensors S1, S2 and S3, S4 are oriented such that a central axis extending through the centres of the sensors in each pair is parallel to the longer dimension of the reflective elements 4, as shown in FIG. 1.

The spacing D1 between the centrepoints of the sensors in each sensor pair 6a, 6b and 6c, 6d, in the direction parallel to the longer dimension of the reflective elements 4 (hereinafter referred to as the x-direction) is given by the equation D1=(2*N1−1)Hₒ, where Hₒ is the length of the longer dimension of the reflective element 4 and N1 is an integer greater than 1.

The spacing D2 between the centrepoints of the sensors in each sensor pair 6a, 6d and 6b, 6c, in a direction normal to the longer dimension of the reflective element (hereinafter referred to as the y-direction) is given by the equation D2=(2*N2−1)Vₒ, where N2 is an integer greater than 1 and Vₒ is the length of the shorter dimension of the reflective elements 4.

The diameter d of each of the sensors 6a, 6b, 6c and 6d must be less than or equal to the spacing V1 between the reflective elements 4 in the y-direction. The spacing between the reflective elements 4 in the x-direction preferably equals the length Hₒ of the longer dimension of the reflective elements 4.

The positional configurations of the sensors 6a, 6b, 6c, and 6d relative to the reflective elements 4 and the substrate 2 are illustrated in FIG. 1. Light sources (not shown) may be integrated to the detectors or deployed separately. The light sources and detectors may be those in an optical disc system.

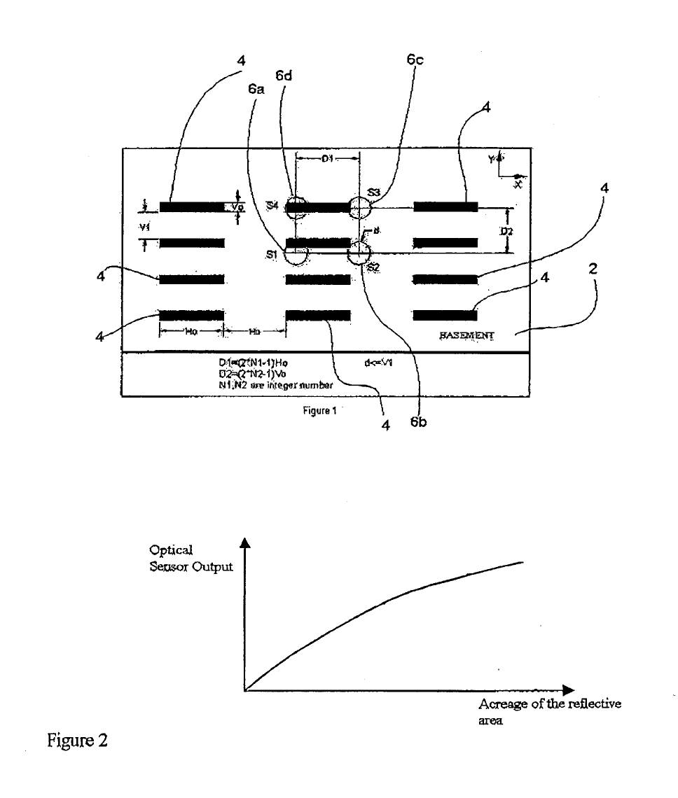

FIG. 2 shows the output, of one of the sensors 6a, 6b, 6c or 6d of FIG. 1 relative to the amount of reflected area seen by that sensor. The output of the sensor increases when the area of the light reflected from the reflective element 4 falling on the sensor is increased.

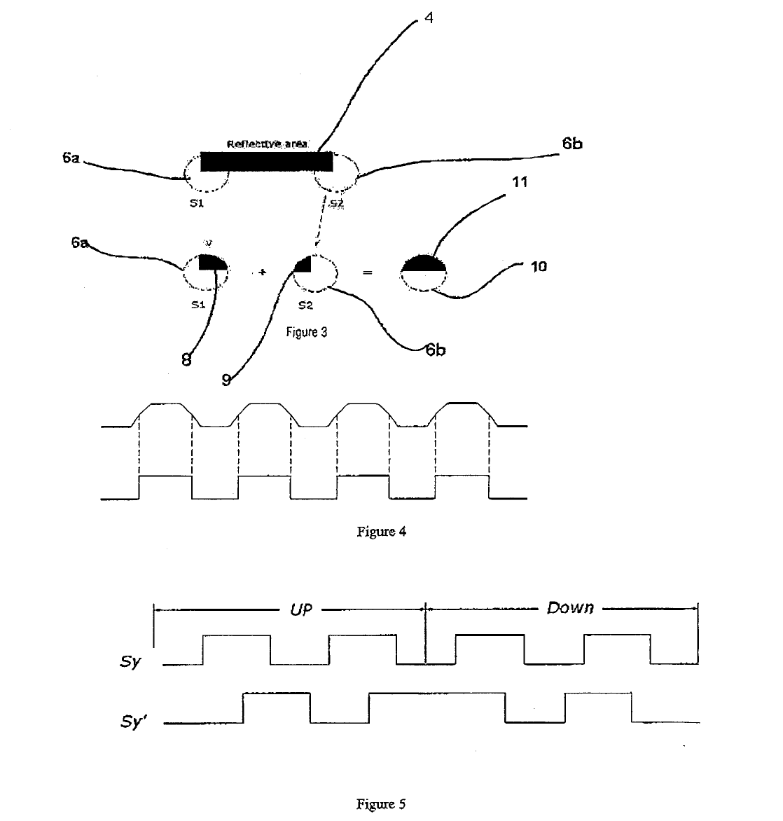

FIG. 3 illustrates the compensatory effect on the output signal of a pair of the sensors 6a, 6b, or 6c, 6d, for translational movement of the image, that is, the substrate 2, in a direction perpendicular to that being measured. As the light 8 reflected from the reflective element 4 falling on the sensor 6a moves from one sensor 6a to its laterally adjacent partner 6b, the output signal from the first sensor 6a decreases and the output signal from the second sensor 6b due to the reflected light 9 falling on it increases proportionally so that the sum of the outputs of the sensors 6a and 6b remains substantially constant irrespective of movement of the substrate in the x-direction. This is equivalent to all of the light 11 falling on one sensor 10, as shown hypothetically in FIG. 3.

FIG. 4 shows the variation of the sum of the output signals of the sensors 6a and 6b in a pair of sensors as the substrate 2 is moved in the y-direction. The upper trace shows the result of adding the output signals of the sensors 6a, 6b, and the lower trace shows the effect of converting the waveform of the upper trace into a rectangular waveform by slicing at the half amplitude level. The amplitude of the motion of the substrate 2 in the y-direction is determined by counting cycles, which corresponds to the number of reflective elements 4 passing the pairs of sensors 6a, 6b in the y-direction.

In FIG. 5, the upper trace shows the rectangular output signal waveforms obtained by addition of the output signals of one laterally adjacent pair of sensors 6a, 6b. The lower trace shows the rectangular output signal waveforms obtained by addition of the output signals of the other pair of laterally adjacent sensors 6c, 6d. By adjusting the spacing of the sensors 6a-6d and the reflective elements 4, such that the output signals of the two pairs of sensors are shifted 90 degrees in phase relative to each other, it is possible to determine the direction of motion of the substrate 2 as well as the amplitude of the motion.

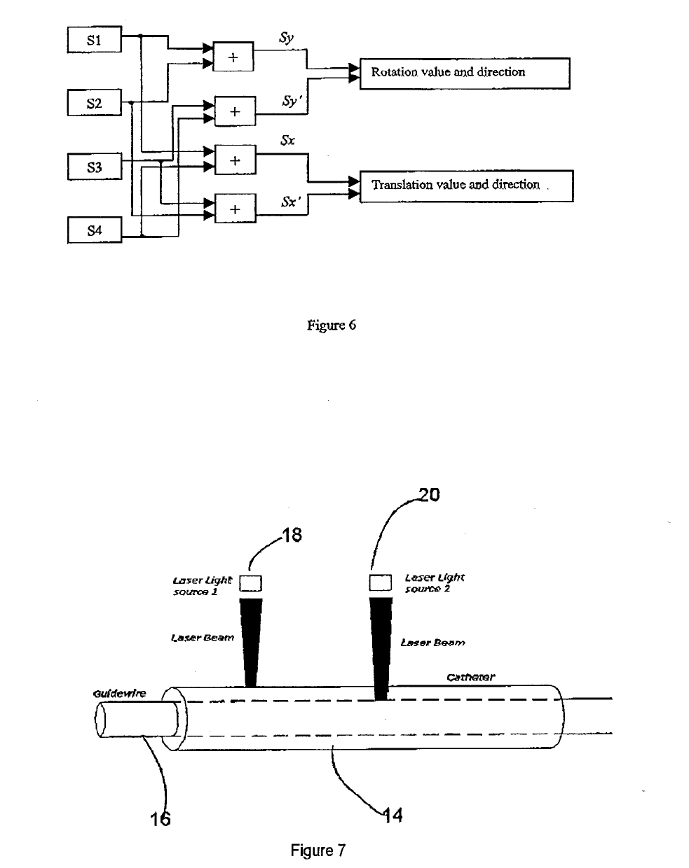

FIG. 6 is a schematic of a system showing the extraction of information of motion of the substrate in two orthogonal directions using the system. This is discussed in more detail below.

FIG. 7 is an embodiment in which the system and method shown in FIGS. 1 to 6 is applied to measure the translation and rotation of a catheter 14 and a guide wire 16 located within the catheter 14. In this embodiment, the substrate 2 carrying the array of reflective elements 4 shown in FIG. 1 comprises the outer coating of the catheter 14 and the outer surface of the guide wire 16. The catheter 14 and the guide wire 16 each carry a set of reflective elements of the type shown in FIG. 1.

The catheter 14 and the guide wire 16 are each illuminated by a laser light source 18, 20. The laser light source 18 illuminates the reflective elements (not shown) on the outer coating of the catheter 14, and light is reflected back to an array of sensors of the type shown in FIGS. 1 and 3, to measure translation and rotation of the catheter 14. Similarly, a second light source 20 illuminates reflective elements (not shown) on the guide wire 16 through the catheter wall 14, which is made of semitransparent material to allow light to pass therethrough. The translation and rotation of the guide wire 16 may be measured independent of the measurement of the translation and rotation of the catheter 14.

In a preferred embodiment, the system may be used to measure motion in two dimensions in the manner described below.

The substrate 2 whose motion is to be measured, carries the array of equally spaced and uniformly aligned reflective elements 4 mounted thereon, as shown in FIG. 1. The reflective elements 4 are illuminated by a light source, preferably a laser, and light reflected from the reflective elements 4 is detected by the array of photosensors 6a, 6b, 6c, 6d. The array of photosensors 6a, 6b, 6c, 6d preferably comprises four sensors S1-S4 forming a rectangular array, the sides of the rectangle being parallel to and perpendicular to the longer dimension of the reflective elements 4.

As the substrate 2 is moved, the beams of light reflected from the reflective elements 4 move across the sensors 6a, 6b, 6c 6d. If the substrate is moved in a direction which causes the reflected light to move across the sensors 6a and 6d, in the y-direction from S1 to S4, as shown in FIGS. 1 and 3, the output signal of a sensor as the beam passes over it will vary from zero to a maximum value. The maximum output signal is obtained when the beam passes across the middle of the sensor and a zero value is obtained either before the beam reaches the sensor or after the beam has cleared the sensor.

In a preferred embodiment, the spacing D1 of adjacent sensors 6a, 6b and 6c, 6d is given by the equation D1=(2N1−)Hₒ, where Hₒ is the length of the reflective element 4 in the longer dimension and N1 is an integer greater than 1. If the width of the reflected beam as it strikes the sensors is equal to D1, and the beam is displaced in the x-direction such that it does not fall on one of the sensors in a sensor pair, thereby reducing the output signal from that sensor, the beam will fall on the other sensor of the pair and the output of that sensor will rise to offset the loss in the first sensor to give a substantially uniform output signal. This is shown in FIGS. 1 and 3. Thus, the sum of the output signals of a pair of sensors S1 and S2 is independent of motion of the substrate 2 in the x-direction A similar output may be obtained by adding the output signals of the other pair of sensors 6c and 6d (S3 and S4).

If the substrate 2 is moved in the y-direction from S1-S4, as shown in FIG. 1, then the sum of the output signals of the sensors 6a, 6b (S1 and S2) will give a waveform varying between a low level and a higher level (see FIG. 4) which may be converted to a rectangular waveform of the same frequency and phase by a comparator (not shown). A similar output may be obtained by adding the output signals of the other pair of sensors 6c and 6d (S3 and S4).

A suitable choice of the spacing of the reflective elements 4 in the y-direction, will result in the two waveforms of the output signals of the pairs of sensors (S1+S2;S3+S4) as shown in FIG. 5, being 90° out of phase. By comparing these two waveforms, the direction of motion of the substrate 2 may be determined (FIG. 5). The same procedure applied to the outputs of sensors 6a and 6d (S1 and S4) and 6b and 6c (S2 and S3) will determine movement in the x-direction, independent of movement in the y-direction.

In the process of detecting translational movement, translational distance is calculated as follows.

Each cycle of the waveform shown in FIG. 4, which illustrates the sum of the output signals of the sensors 6a and 6b (S1 and S2), represents a movement of the substrate 2 in the y-direction of (Vₒ+V1) where Vₒ is the length of the shorter dimension of the reflective elements 4, and V1 is the spacing V1 between the reflective elements 4 in the y-direction. Thus, by counting the cycles, it is possible to determine the displacement of the substrate 2 in the y-direction. The movement Mγ in the y-direction may be calculated according to the equation:

M_{y}=m*(Vₒ+V1)

where m is the number of cycles counted.

The movement of the substrate 2 in the x-direction may be calculated using the sum of output signals of the sensors 6a and 6d (S1 and S4). One cycle in this waveform (not shown) corresponds to 2 Hₒ, where Hₒ is the length of the longer dimension of the reflective elements 4, as shown in FIG. 1. Thus, by counting the cycles, it is possible to determine the displacement of the substrate 2 in the x-direction. The movement Mₓ in the x-direction may be calculated according to the equation:

Mₓ=2 nHₒ

where n is the number of the period of the sum of the output signals of the sensors 6a and 6d (S1 and S4).

As shown in FIG. 5, S_{y} is the sum of the output signals of the sensors 6a, 6b (S1 and S2) and S_{y}′ is the sum of output signals of the sensors 6d and 6c (S4 and S3), Both signals S_{y} and S_{y}′ correspond to the motion of the substrate 2 in the y-direction independent of the motion of the substrate 2 in the x-direction and there is 90° phase difference between the signals, due to the spacing of the reflective elements 4 relative to the spacing of the sensors 6a-6d, as shown in FIGS. 1 and 5. When the substrate moves upwards in the y-direction, that is, Mγ is positive, S_{y} leads S_{y}′ by 90°. When the substrate 2 moves down in the y-direction, that is, Mγ is negative, S_{y}′ leads S_{y} by 90°. Therefore, from the two waveforms shown in FIG. 5, the direction of motion of the substrate 2 in the y-direction may be determined using conventional techniques, for example, as used in an optical encoder.

In the same way, the direction of motion of the substrate 2 in the x-direction may be determined using Sₓ and Sₓ′ where Sₓ is the sum of the output signals of the sensors 6a and 6d (S1 and S4) and Sₓ′ is the sum of the output signals of the sensors 6b and 6c (S2 and S3).

In a preferred embodiment, the system may be applied to a catheter 14 and its enclosed guide wire 16 (see FIG. 7). In this embodiment, x-motion corresponds to a translation of the catheter 14 or the guide wire 16 and y-motion corresponds to rotation thereof. In operation, light preferably from a laser source 18, illuminates reflective elements (not shown) on the outer surface of the catheter 14 and the reflected light is collected by an array of sensors (not shown) which may be integral with or separate from the laser source 18. A similar configuration of light source 20 illuminates reflective elements (not shown) on the guide wire 16 through the catheter 14 which is made of semitransparent material, to detect translation and rotation of the system.

Such a system may be used to control the motion of the catheter 14 and guide wire 16 in an interventional radiology simulator or an interventional radiology remote operation system. A schematic of this application is illustrated in FIG. 6. The reflective elements are located on the outer surface of the catheter 14 and the guide wire 16. In order to navigate the motion of the guide wire 16 inserted inside the catheter 16, the catheter 16 is preferably made of semi-transparent material. As shown in FIG. 7, the focus planes of the laser light sources 18 and 20 for the catheter 14 and guide wire 16 respectively, are separately positioned on the catheter and guide wire. The sensors may be colour sensitive, for example, the sensors for the catheter 14 may be sensitive to red colour and the sensors for the guide wire 16 may be sensitive to blue colour. By marking the reflective areas in different colours, the motions of several catheters and the guide wires may be tracked with no interference.

The translational and rotational movements of the catheter 14 and guide wire 16 may be calculated as described above with respect to FIGS. 1 to 6.

Various alternatives to the embodiments described above may be made, for example, whilst the embodiments have been described with reference to the use of a catheter and guide wire in interventional radiology (IR) procedures, the above method and system may also be used in other applications where two-dimensional motion tracking is required, such as in a computer mouse, microinjection devices for transgenic work, or some industry applications. Furthermore, although in the embodiments described above the use of multiple reflective elements is envisaged, the invention is not limited in this respect, and the invention may alternatively employ only a single reflective element. Such a technique may exploit the fact that the reflective element has non-zero length

In a preferred embodiment, the precision of the optical tracking is determined by the radius of the laser spot. The laser beam may be focussed to a spot with a radius of approximately 0.5 μm. As an example, the laser detector used in a second generation phased-DVD disc system is a blue laser with a spot radius of 400 to 450 mm. The track pitch may be 0.37 μm. Thus, this size of reflective area may be realised in industry.

In summary, an embodiment of the present invention is directed to an optical method of tracking the translational and rotational motion of catheters and guide wires in interventional radiology procedures. With this method, the motions of the catheters and guide wires may be navigated using one tracking unit. The precision of the optical tracking is preferably determined by the radius of the laser light source. The tracking unit may be based on the optical method described above and may act as one of the key components in interventional radiology simulation systems and interventional radiology remote operation systems. In this way the system is simplified over prior art systems. Optical sensors may navigate the motions of all of the catheters and guide wires and the motion relationship between the catheter and the guide wire may remain the same (the guide wire is inside the catheter). Furthermore, the mechanical structure may be of a small size. Also, the tracking resolution may be increased to the level of micrometres and the length of the catheters and the guide wires need not be modified.

A further advantage of a preferred embodiment of the invention is that the guide wire and catheter may exist in the same housing for movement tracking purposes. In such an embodiment, the catheter is preferably transparent to allow the second light source to be reflected off the internal guide wire. If several different light sources are used, the motions of several catheters and guide wires may be tracked simultaneously.

Furthermore, in a preferred embodiment of the invention, there is no signal loss regardless of how much motion is experienced by the object being tracked.

1. A method for detecting motion of a body in a number of dimensions comprising the steps of:
(a) attaching an array of one or more reflective elements to said body such that said reflective elements move with said body;
(b) illuminating said array of reflective elements with light from a light source;
(c) sensing said light reflected from said array of reflected elements with an array of sensors for generating output signals corresponding to motion of said body; and
(d) processing said output signals to determine motion of said body in a number of dimensions.

2. A method according to claim 1, wherein said step of processing said output signals comprises determining amplitude and direction of said motion of said body.

3. A method according to claim 1, wherein said step of sensing said light from said reflected elements with an array of sensors comprises providing an array of four sensors.

4. A method according to claim 1 wherein the step of attaching an array of one or more reflective elements to said body such that said reflective elements move with said body comprises mounting said reflective elements on said body.

5. A method according to claim 1 wherein the step of attaching an array of one or more reflective elements to said body such that said reflective elements move with said body comprises integrally forming said reflective elements with said body.

6. A method according to claim 1, wherein the step of attaching an array of reflective elements comprises attaching an array of reflective elements having a rectangular periphery.

7. A method according to claim 1, wherein the step of attaching an array of reflective elements comprises attaching said reflective elements to form a regularly spaced array of rows and columns of reflective elements of equal size.

8. A method according to claim 1, wherein said step of sensing said light from said reflected elements with an array of sensors comprises providing an array of four sensors arranged in a rectangular pattern with one sensor in each corner.

9. A method according to claim 1, wherein said reflective elements have a longitudinal axis and a length along said longitudinal axis, and said step of attaching an array of one or more reflective elements to said body further comprising arranging said reflective elements in an array with a space along said longitudinal axis between each reflective element in said array, said space being substantially equal in length to said length of said reflective elements along said longitudinal axis.

10. A method according to claim 9, further comprising arranging said sensors in said array such that said sensors are separated from each other by a space having a length D1 along said longitudinal axis, the method further comprising the step of selecting said length of said space according to the formula:
D1=(2*N1−1)Hₒ, where N1 is an integer greater than 1 and Hₒ is said length of each of said reflective elements along said longitudinal axis, to compensate for displacement of said body along said longitudinal axis.

11. A method according to claim 9, wherein said sensors in said array of sensors have a diameter, and wherein said step of attaching an array of one or more reflective elements comprises attaching said reflective elements such that they are separated from each other in said array in a direction normal to said longitudinal axis by a space having a height, said height of said space being greater than or equal to said diameter of said sensors.

12. A method according to claim 9, wherein the reflective elements have a width Vₒ, and said sensors are separated from each other in said array of sensors by a distance D2 in a direction normal to the longitudinal axis of the reflective elements, said method further comprising the step of arranging said sensors in said array of sensors such that D2 is given by the equation:
D2=2*(N2−1)Vₒ
where N2 is an integer greater than 1 and Vₒ is the width of said reflective elements.

13. A method according to claim 12, wherein said method further comprises adjusting said distance D2 between sensors in said array of sensors in a direction normal to the longitudinal axis of the reflective elements to alter the phase of said output signals of said sensors spaced in a direction normal to the longitudinal axis.

14. A method according to claim 13, wherein adjusting said distance sets a phase shift between said output signals of said sensors spaced in a direction normal to the longitudinal axis to approximately 90 degrees to determine direction of movement of said body.

15. A method according to claim 12, wherein said step of processing said output signals to determine motion of said body comprises determining distance moved by said body in a first dimension using the formula:
M₁=m*(V₀+V1)
where M₁ is the distance moved by said body and said reflective elements in a first dimension, m is the number of said reflective elements passing over said sensors due to said distance to be measured, Vₒ is width of the reflective elements and V1 is the spacing between the reflective elements in a direction normal to the longitudinal axis.

16. A method according to claim 12, wherein said step of processing said output signals to determine motion of said body comprises determining distance moved by said body in a second dimension using the formula:
M₂=2 nHₒ
where M₂ is displacement of said body and said reflective elements in a second dimension, n is the number of said reflective elements passing over said sensors due to said distance to be measured, and Hₒ is the length of the reflective element in said longitudinal direction.

17. A method according to claim 1, wherein said step of processing said output signals to determine motion of said body comprises determining direction of displacement of said body by comparing the phase of said output signals of said sensors.

18. A method according to claim 1, wherein the step of sensing said light reflected from said array of reflected elements with an array of sensors comprises using sensors sensitive to illuminations of different wavelengths.

19. A method according to claim 1, wherein said method is an optical tracking method.

20. A method of determining motion of a catheter and/or a guide wire comprising the method steps of claim 1.

21. A method of determining motion of a catheter and/or a guide wire in interventional radiology (IR) procedures comprising the method steps of claim 1.

22. A method according to claim 20, wherein said step of sensing said light from said reflected elements with an array of sensors comprises providing an array of four sensors per body whose motion is to be detected.

23. A method of determining motion of a catheter and a guide wire mountable in a common housing comprising the method steps of claim 1.

24. A method according to claim 23, wherein the method further comprises locating said guide wire within said catheter, said catheter being transparent or semi-transparent to allow a second light source to be reflected from said guide wire.

25. A method according to claim 1, wherein said step of attaching one or more reflective elements comprises attaching an array to each of a plurality of bodies, said method further comprising illuminating each array with a different light source for detecting motion of a plurality of bodies.

26. A system for detecting motion of a body in a number of dimensions comprising:
(a) an array of one or more reflective elements attachable to said body such that said reflective elements move with said body;
(b) an array of sensors for sensing light reflected from said array of reflective elements on illumination of said reflective elements by a light source, said sensors being adapted to generate output signals corresponding to motion of said body; and
(c) a processor for processing said output signals to determine motion of said body in a number of dimensions.

27. A system according to claim 26, wherein said processor is arranged to determine amplitude and direction of said motion of said body.

28. A system according to claim 26, wherein said array of sensors comprises four sensors.

29. A system according to claim 26, wherein said reflective elements are mountable on said body.

30. A system according to claim 26, wherein said reflective elements are integrally formed with said body.

31. A system according to claim 26, wherein each of said reflective elements has a rectangular periphery.

32. A system according to claim 26, wherein said reflective elements are arranged to form a regularly spaced array of rows and columns of reflective elements of equal size.

33. A system according to claim 26, wherein said array of sensors compnses an array of four sensors arranged in a rectangular pattern with one sensor in each corner.

34. A system according to claim 26, wherein said system is adapted for use in an optical tracking method.

35. A system according to claim 26, wherein said body is a catheter and/or a guide wire.

36. A system according to claim 26, wherein said body is a catheter and/or guide wire for use in interventional radiology (IR) procedures.

37. A system according to claim 26, wherein said reflective elements have a longitudinal axis and a length along said longitudinal axis, said reflective elements being arranged in said array with a space along said longitudinal axis between each reflective element in said array, said space being substantially equal in length to said length of said reflective elements along said longitudinal axis.

38. A system according to claim 37, wherein said sensors are separated from each other by a space having a length D1 along said longitudinal axis, the method further comprising the step of selecting said length of said space according to the formula:
D1=(2*N1−1)Hₒ, where N1 is an integer greater than 1 and Hₒ is said length of each of said reflective elements along said longitudinal axis, to compensate for displacement of said body along said longitudinal axis.

39. A system according to claim 37, wherein said sensors in said array of sensors have a diameter, and wherein said reflective elements are arranged such that they are separated from each other in said array in a direction normal to said longitudinal axis by a space having a height, said height of said space being greater than or equal to said diameter of said sensors.

40. A system according to claim 37, wherein the reflective elements have a width Vₒ, and said sensors are separated from each other in said array of sensors by a distance D2 in a direction normal to the longitudinal axis of the reflective elements, said sensors being arranged in said array of sensors such that D2 is given by the equation:
D2=2*(N2−1)Vₒ
where N2 is an integer greater than 1 and Vₒ is the width of said reflective elements.

41. A system according to claim 40, wherein said system further comprises adjusting said distance D2 between sensors in said array of sensors in a direction normal to the longitudinal axis of the reflective elements to alter the phase of said output signals of said sensors spaced in a direction normal to the longitudinal axis.

42. A system according to claim 41, wherein a phase shift between said output signals of said sensors spaced in a direction normal to the longitudinal axis is set to approximately 90 by adjusting said distance to determine direction of movement of said body.

43. A system according to claim 40, wherein said processor is arranged to determine distance moved by said body in a first dimension using the formula:
M₁=m*(V₀+V1)
where M₁ is the distance moved by said body and said reflective elements in a first dimension, m is the number of said reflective elements passing over said sensors due to said distance to be measured, V₀ is width of the reflective elements and V1 is the spacing between the reflective elements in a direction normal to the longitudinal axis.

44. A system according to claim 40, wherein said processor is arranged to determine distance moved by said body in a second dimension using the formula:
M₂=2 nHₒ
where M₂ is displacement of said body and said reflective elements in a second dimension, n is the number of said reflective elements passing over said sensors due to said distance to be measured, and Hₒ is the length of the reflective element. In said longitudinal direction.

45. A system according to claim 41, wherein said processor is arranged to determine direction of displacement of said body by comparing the phase of said output signals of said sensors.

46. A system according to claim 26, wherein said sensors comprise one or more sensors sensitive to illuminations of different wavelengths.

47. A system according to claim 26, comprising an array of four sensors per body whose motion is to be detected.

48. A system of determining motion of a catheter and a guide wire mountable in a common housing comprising the system of claim 26.

49. A system according to claim 48, wherein said guide wire is locatable within said catheter, said catheter being transparent or semi-transparent to allow a second light source to be reflected from said guide wire.

50. A system according to claim 26, wherein an array of one or more reflective elements is attachable to each of a plurality of bodies, said reflective elements of each array being illuminated in use with a different light source for detecting motion of a plurality of bodies.
